(19) United States
(12) Patent Application Publication
(10) Pub. No.: US 2006/0276843 A1
(43) Pub. Date: Dec. 7, 2006

(54) Life energy device

(76) Inventor: Mao-Chun Chen, Taipei (TW)

Correspondence Address: Mao-Chun Chen, P.O. Box 26-757, Taipei 106, TW

(21) Appl. No.: 11/142,217

(22) Filed: Jun. 2, 2005

(51) Int. Cl. A61N 1/00

(52) U.S. Cl. 607/2

(57) ABSTRACT

A life energy device includes an ore and a life energy unit, wherein the ore can absorb and release an energy ranged in a predetermined frequency band and the life energy unit includes a circuit board mounted in the ore, a chip mounted on the circuit board and having a circuit design for magnifying the energy, and a drive unit mounted on the circuit board for driving the chip, thereby the ore can absorb slight energies in the natural environment and then magnify the range of the energy through the chip so as to form a magnetic field therearound, and thus, the cells of human body can be filled with energy through contacting therewith the ore so as to activate the cells and improve a constitution of the human body.

### BACKGROUND OF THE INVENTION

1. Field of the Invention

The present invention relates to a life energy device, and more particularly to a device for releasing life energy.

2. Description of the Related Art

All of the organism are composed by cells. Single cell organism is for example bacterium whose body is composed by one single cell. Multi-cells organism like the plant and animal whose body is composed by hundreds of millions cells, hence the cell is a basic component of the organism. Generally, electric charges are existed inside and outside a normal cell, and the physiological function of the cell is easily influenced by electromagnetic field. The chemical reaction in cell usually contains electrons transferred, and the unsaturated fatty acid in cell is more easily changed by light and electromagnetic field, and further influences the physiology of the cell. The fresh cells have active metabolism, and their electrons capacity are more than normal condition. When the cells are aged, their electrons capacity reduce and simultaneously their activity decreases. Based on the Rule of Right Hand theory, an energized cell may produce electricity because the conductor thereof is tangent to the magnetic field so as to keep the cell in an active status.

Due to the custom of the diet and life environment changed, people often eat much deep-fried food. The stress makes people imbalance, and it induces much free radicals produced in human body. The free radicals have strong toxicity to cells, and they interfere normal metabolism in the body. If our bodies are in this situation for a long time, it forces the cells quickly fall into aged status or mutate themselves. One of the possible reasons about the cancer cells growth is normal cells fall into stress environment and transfer to mutant. If we supply the life energy to the cells, it may activate the cells and strengthen the immuno-system. It makes cells have better tolerance in bad environment, and makes human healthy.

Consequently, because of the technical defects of described above, the applicant keeps on carving unflaggingly through wholehearted experience and research to develop the present invention, which can effectively overcome the problems described above.

### SUMMARY OF THE INVENTION

Accordingly, the primary objective of the invention is to provide a life energy device, which keeps the life energy and effectively and stably releases the life energy. The life energy has functions of activated cells and strengthened immuno-system.

Another objective of the present invention is to provide a life energy device including a material which can naturally vibrate for producing stable vibration frequency, and the material may stably release the life energy using the stable frequency thereof.

The present invention provides a life energy device including an ore and a life energy unit, wherein the ore can absorb and release an energy ranged in a predetermined frequency band and the life energy unit includes a circuit board mounted in the ore, a chip mounted on the circuit board and having a circuit design for magnifying the energy, and a drive unit mounted on the circuit board for driving the chip, thereby the ore can absorb slight energies in the natural environment and then magnify the range of the energy through the chip so as to form a magnetic field therearound, and thus, the cells of human body can be filled with energy through contacting therewith the ore so as to activate the cells and improve a constitution of the human body.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 is a three-dimensional drawing illustrating a life energy device according to the present invention;

FIG. 2 is a block diagram showing a chip of the life energy device according to the present invention;

FIG. 3 is a schematic view illustrating the operation of the life energy device according to the present invention; and

FIG. 4 is a three-dimensional drawing illustrating another embodiment of the life energy device according to the present invention.

### DETAILED DESCRIPTION OF THE PREFERRED EMBODIMENT

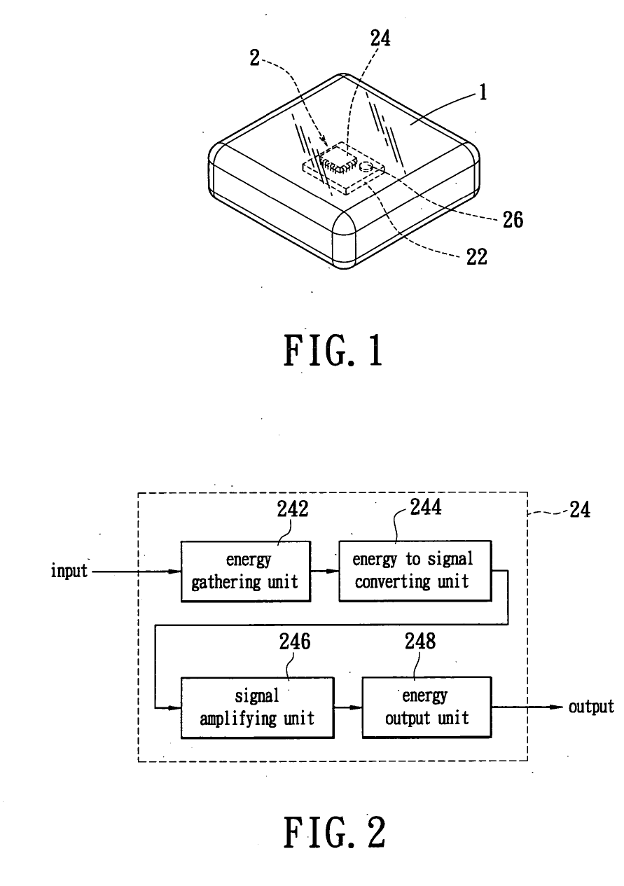

Please refer to FIGS. 1-4. The present invention provides a life energy device including an ore 1 which can naturally vibrate to produce a stable vibration frequency for absorbing and releasing an energy ranged in a predetermined frequency band, and including a life energy unit 2 which comprises a circuit board 22 mounted in the ore 1, a chip 24 mounted on the circuit board 22 and having a circuit design for magnifying the energy, and a drive unit 26 mounted on the circuit board 22 for driving the chip, thereby the ore 1 may absorb the slight energy in the natural environment and then magnify the range thereof through the chip 2 so as to form a magnetic field therearound, and thus, the cells of human body may be filled with energy through contacting with the ore so as to activate the cells and improve a constitution of the human body. Moreover, the ore 1 is made of silicon dioxide, for example the crystal, or made of diamond so that the ore 1 may produce a natural vibration with a stable frequency for absorbing and stably releasing a life energy 3. And, the life energy 3 can penetrate a normal insulating object and also the circulatory system of human body for energizing billions of cells inside the body so that it owns the ability of activating blood cells and hormones. When under using, the life energy device should be placed near the human body so that the life energy 3 released by the life energy device which is in form of field magnetic energy and is slowly and stably released can directly penetrate the human body for energizing the cells and directly improving the cell condition. Furthermore, through activating the cells, the toxicity, for example, carbon dioxide, sulfide dioxide and radicals, inside the body can be rapidly metabolized and discharged from the body so as to improve body health.

The chip 24 includes an energy gathering unit 242 for gathering the slight energy in the natural environment, an energy to signal converting unit 244 for converting the gathered energy, a signal amplifying unit 246 for amplifying the converted signal, and an energy output unit 248 for outputting the signal, wherein the energy gathering unit 242 can gather energies in different forms, such as electric waves, heat energy, or magnetic waves. In addition, when the material of the chip 24 is similar to or equivalent to the ore 1, the stable vibration frequency produced by the chip 24 might resonate with the ore 1 and therefore further magnify the energy signal.

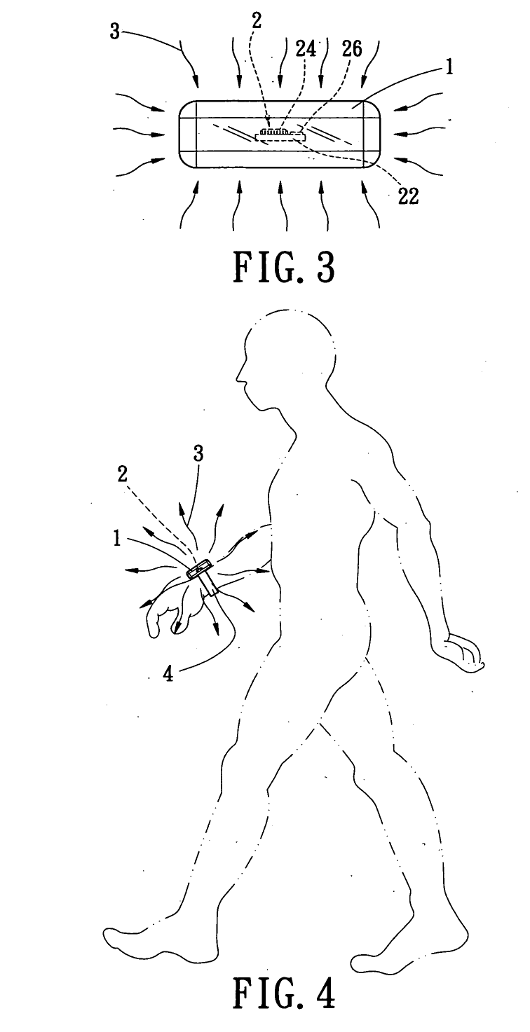

Please refer to FIG. 4, wherein a soft material 4 is further mounted at the ore 1 for comfortably closing to the human body. The soft material 4 can be cotton, flax, wool, silk, artificial silk, nylon, polyester, acrylic fiber, cellulose acetate, cellulose triacetate, flexible fiber, glass fiber, metallic fiber, rubber fiber, flexible plastic, and non woven material etc.

The life energy device can adsorb life energy from outside and release the life energy after filling with the life energy. When we use it, we put the life energy device near the human body, and said device stably releases life energy automatically. The effect period of the life energy device will keep more than one year. Some useful effects for human listed below
1 Improve the inner circulatory system.
2 The blood in blood capillary or the lymth in lymth node is activated for activating cells.
3 The toxicity produced by the cells is accelerated to drain out.
4 Normalize the enzymes produced in the cells for decomposing the toxic compound.
5 Balance the unbalanced electric charge, and decrease the free radicals produced.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A life energy device, comprising:
an ore performing a natural vibration for producing a stable vibration frequency to absorb and release an energy ranged in a predetermined frequency band;
a life energy unit including a circuit board mounted in the ore, a chip mounted on the circuit board and having a circuit design for magnifying the energy, and a drive unit mounted on the circuit board for driving the chip;
thereby the ore absorbs slight energies in the natural environment and then magnifies the range of the energy through the chip so as to form a magnetic field therearound, and thus, the cells of human body are filled with energy through contacting therewith the ore so as to activate the cells and improve a constitution of the human body.

2. The life energy device as claimed in claim 1, wherein the chip comprises an energy gathering unit, a signal amplifying unit, and an energy output unit.

3. The life energy device as claimed in claim 1, wherein the energy gathering unit gathers electric waves, heat energy or magnetic waves.

4. The life energy device as claimed in claim 3, wherein the chip further comprises an energy to signal converting unit for converting the energy into a signal so as to facilitate a sequential process by the signal amplifying unit.

5. The life energy device as claimed in claim 1, wherein the ore is made of silicon dioxide.

6. The life energy device as claimed in claim 5, wherein the silicon dioxide is crystal.

7. The life energy device as claimed in claim 1, wherein the ore is diamond.

8. The life energy device as claimed in claim 1, further comprising a soft material mounted at the ore for comfortably closing to the human body.

9. The life energy device as claimed in claim 9, wherein the soft material is cotton, flax, wool, silk, artificial silk, nylon, polyester, acrylic fiber, cellulose acetate, cellulose triacetate, flexible fiber, glass fiber, metallic fiber, rubber fiber, flexible plastic, or non woven material.
